(19) United States
(12) Patent Application Publication
(10) Pub. No.: US 2014/0155944 A1
(43) Pub. Date: Jun. 5, 2014

(54) ATRAUMATIC FASTENER AND BONE STABILIZATION SYSTEM AND METHOD OF USE

(71) Applicant: Mari S. Truman, Warsaw, IN (US)

(72) Inventor: Mari S. Truman, Warsaw, IN (US)

(21) Appl. No.: 13/895,158

(22) Filed: May 15, 2013

Related U.S. Application Data
(63) Continuation of application No. 12/541,106, filed on Aug. 13, 2009, now abandoned.
(60) Provisional application No. 61/088,445, filed on Aug. 13, 2008.

(51) Int. Cl. A61B 17/86

(52) U.S. Cl. CPC A61B 17/8685
USPC 606/324; 606/328

(57) ABSTRACT

Atraumatic fasteners involving flexible membranes and tensioning means can be used in conjunction with one or more plates, other fastener types of other materials, particularly locked cannulated screws-plate interfaces providing improved capture and restraint of articular and/or juxta-articular bone fragments. A method of securing bone fragments includes securing or capturing a fragment, at least in part, with an atraumatic foldable and/or expandable device which can be inserted “inside out”, that is, the expandable form is inserted through a channel which has been created in the bone or tissue, from the opposite side of the bone or tissue fragment.

### FIELD OF THE INVENTION

The invention relates to a fastener system for internal stabilization of a fractured bone, and more particularly to atraumatic fasteners having flexible membranes and tensioning means as well as an inflatable balloon-like soft catheter for opening passages and pulling pieces of bone together.

### BACKGROUND OF THE INVENTION

Osteoporosis, avascular necrosis and bone cancer are diseases of bone that weaken the bone, predisposing fracture. There are 2 million fractures each year in the United States, of which about 1.3 million are caused by osteoporosis. Avascular necrosis and bone cancers are rarer but can cause bone problems that are currently poorly addressed

It is a common requirement in orthopedic surgical procedures to anchor two or more elements together, such as pieces of a bone, two or more bones, or a combination of soft tissue and bone. This has been accomplished by a number of devices, such as bone bolts that penetrate two pieces of bone and use a nut to draw the segments together, bone screws and interconnecting plates, wires circling at least two pieces of bone, or sutures into the tissue. Often such devices require a relatively large access opening through surrounding and/or covering tissue to implant the anchoring devices. The enlarged access site can increase patient pain and lengthen recovery time.

Surgeons continue to experience problems achieving rigid or stable fixation when trying to capture and stabilize bone regions comprised of thin cortical bone and low strength cancellous bone as is found in juxta-articular bone regions (epiphyseal regions) as found near joints (including the spine, small wrist bones, and ankle), or metaphaseal bone regions, and in osteoporotic bone.

There are currently many methods used to capture these fragments, each with advantages and disadvantages. Some examples of these methods include screws with or with our washers and plates, bolts with or without washers and plates, wires, cables, plates with screws, multiple Plates with screws, plates with screws and cables or wires, plates with cables or wires alone (no screws or bolts), external fixators with pins or k-wires with or without threads (pins or wires through skin have increased risk of pin tract infection), and internal cord fixation device (U.S. Pat. No. 6,368,326 taken et al).

Injection of bone cement (polymethylmethacryate) or partially incorporable calcium phosphate ceramic cements (typically containing tricalcium phosphate and/or hydroxyapatite ceramics) into the cancellous bone region to strengthen the hold internally. More recently Kyphon combined this with an inflatable delivery device functioning in a manner similar to vascular catheters (7156861, 7153306, 6989341, 66425213, for example) which use cements and inflatable devices to restore bone shape and strength, particularly in the spinal vertebral bodies.

### Balloon Catheters

Tissue repair products need to support tissues until the in vivo repair mechanisms have stabilized the tissues (e.g. callus formation in bone healing). While this level of repair can occur in a matter of weeks for young healthy individuals, it can require several months under compromised healing conditions, for example vascular compromise, elderly, disease, etc. A tissue repair device should maintain mechanical integrity to assure that fragments remain in position for about 6 months, and preferably for 1 year to assure the desired outcome in the face of delayed union or non-union.

A balloon catheter is a type of “soft” catheter with an inflatable “balloon” at its tip that is used during a catheterization procedure to enlarge a narrow opening or passage within the body. The balloon comprises an inflatable, non-expandable balloon body for insertion into the bone. In the prior art, the balloon is used to reshape and reposition bone. The inflation of the inflatable device causes a compacting of the cancellous bone and bone marrow against the inner surface of the cortical wall of the bone to further enlarge the cavity or passage. The inflatable device is then deflated and then is completely removed from the bone. A smaller inflatable device, a starter balloon, can be used initially, if needed, to initiate the compacting of the bone marrow and to commence the formation of the cavity or passage in the cancellous bone and marrow. After this has occurred, a larger, inflatable device is inserted into the cavity or passage to further compact the bone marrow in all directions. An improved method of constructing the shape and size of inflatable devices for use with the foregoing apparatus and method was revealed in U.S. Pat. No. 5,972,015.

In U.S. Pat. No. 7,153,306, a method using expandable devices is shown in which two long bone fragments are drawn together and held from within the bone cavity by compressing cancellous bone an filling the intramedullary canal.

In U.S. Pat. Nos. 4,969,888 and 5,108,404, the balloon body has a shape and size to compress at least a portion of the cancellous bone to form a cavity in the cancellous bone and to restore the original position of the outer cortical bone, if fractured or collapsed. The balloon is prevented from applying excessive pressure to the outer cortical bone. The wall or walls of the balloon are such that proper inflation the balloon body is achieved to provide for optimum compression of all the bone marrow. The balloon is able to be folded so that it can be inserted quickly into a bone. The balloon can be made to have a suction catheter.

In U.S. Pat. No. 7,153,306, an intramedullary device used to secure two ends of along bone is disclosed.

The devices disclosed were not configured to provide supplemental fixation in the manner that traditional trauma devices such as screws, plates and suture buttons are currently used. The disclosed system provides the surgeon with new tools that can be used to realign and reattach bone and soft tissue fragments The apparatus and method of the present invention are especially suitable for use in the fixation of, but not limited to, vertebral body compression fractures, Colles fractures, and fractures of the proximal humerus.

### DISADVANTAGES IN PRIOR ART

Internal Fixation: In thin cortical bone and weak cancellous bone the holding power of a screw thread is directly proportional to the strength of the bone materials, the outer diameter of the screw and the thread engagement distance. In the case of thin cortices, where the screw threads are primarily engaged in weak cancellous bone, the holding power of the screw is frequently not sufficient to stabilize fracture fragments under post-operative load regimes. Similarly, small wires, or cable, apply concentrated forces and higher pressures that effectively cut through these weaker bone fragments.

Percutaneous Fixation: External fixation pins, wires or screws pierce through the skin and other tissues, increasing the risk of infection. Additionally, when placement cannot avoid muscle compartments, percutaneous fixation can restrict range of motion.

Fragment Instability: When the implant-bone construct is not stable under appropriate post-operative rehabilitation conditions, bracing and casting are needed to prevent motion and reduce loads. Immobilization results in tendon adhesions, and tendon/muscle and ligament contraction, which causes stiffness and functional motion loss.

Many of the previous solutions include mechanical fasteners, such as toggle bolts, nuts, secondary plates, grapple hooks, etc., manufactured out of solid materials that remain proud relative to a bone surface. Even when sharp edges are removed, the material that protrudes above the bone can irritate overlying tissues, particularly frequently moving structures such as tendons and ligaments.

Bonutti (U.S. Pat. No. 6,997,940—FIG. 44) previously patented the generic use of traditional sutures and buttons for fracture fixation. The disadvantage of the Bonutti devices are that larger incision or multiple (dual) incisions are required to insert and deploy the devices, and the sutures are necessary independent and flexible structures incapable of imparting rigidity in bending, and incapable of provision of a buttressing function in conjunction with a plate in the face of physiologic loading and severe comminution.

Patents that describe related materials include US pending application, 2004-0077739, U.S. Pat. No. 5,296,518 1994, U.S. Pat. No. 5,250,649 1993, WO, A2, 9947097, U.S. Pat. No. 6,713,568 2004, and PCT/SE01/02893 02051920

Publications relating to the material and the products it is being used for include:
Nilsson A, Liljensten E, et al, (2005). “Results from a degradable TMC joint spacer (Melon) compared with tendon arthroplasty.” J Hand Surg [Am] 30A:380-389. Abstract at PubMed
Gretzer C, et al. (2004). “Changes of inflammatory cell influx and cytokines during transition from acute inflammation to fibrous repair around implanted materials.” 7th World Biomaterials Congress, Sydney. (In press)
Gretzer, C., et al, (2003). “Adhesion, apoptosis and cytokine release of human mononuclear cells cultured on degradable poly(urethane urea), polystyrene and titanium in vitro.” Biomaterials 24(17): 2843-52. Abstract at PubMed
Liljensten E, et al. (2002). “Studies of polyurethane urea bands for ACL reconstruction.” J Mat Sci: Mat in Medicine 13(4): 351-359 Abstract at PubMed
Gisselfalt, K., et al. (2002). “Synthesis and properties of degradable poly(urethane urea)s to be used for ligament reconstructions.” Biomacromolecules 3(5): 951-8. Abstract at PubMed
Gisselfält K and Flodin P (1998). “A biodegradable material for ACL reconstruction.” Macromol Symp 130: 103-111.

In U.S. Pat. Nos. 4,969,888 5,108,404, and 7,153,306, an apparatus and method are disclosed for the fixation of fractures or other conditions of human and other animal bone systems, both osteoporotic and non-osteoporotic using a balloon catheter.

Restoration of normal joint function, particularly in the hand and foot, following tendon laceration requires reestablishment not only of the continuity of the tendon fibers, but also of the gliding mechanism between the tendon and its surrounding structures. Like many other tissues, tendons heal by deposition of scar tissue at the site of injury. While the initial formation of scar tissue between the tendon ends provides physical continuity at the site of the disruption, proliferation of the scar tissue between the tendon and adjacent tissues is undesirable, indeed harmful, because these attachments impede tendon gliding, which is of critical importance for function. Thus, unlike the repair of other musculoskeletal tissues, which can fail because of too little healing, a repair of a lacerated tendon can also fail because of too much healing: adhesions result in loss of motion, contracture formation, and functional disability.

Chemical modulation has been attempted to diminish the amount of scar formation after repair. The chemical agents that have been used in these efforts include local and parenteral corticosteroids, dimethyl sulfoxide, beta-aminoproprionitrile, hyaluronic acid, and 5-fluorouracil, among many others. The common principle of these methodologies is reduction of inflammation. In the case of the corticosteroids and hyaluronic acid, the goal is to diminish inflammation by inhibiting lymphocyte migration, proliferation, and chemotaxis as well as macrophage motility. Similarly, 5-fluorouracil, an antimetabolite, suppresses scar formation by inhibiting contraction of collagen lattice and proliferation of inflammatory cells.

Use of porous woven biocompatible, degradable, inert fabrics in the fastener cause fewer irritations and ruptures and, the preferred embodiments will eventually break down by hydrolysis without generating local acidity.

### SUMMARY OF THE INVENTION

The disclosed biodegradable or bio-incorporable fasteners provide improved capture and restraint of peri-articular and/or juxta-articular bone, or bone and soft tissue fragments, and includes the use of tension-band flexible plates.

The system of the present invention facilitates restoration of early range of motion and function by capturing bone fragments using flexible materials that can be deployed or activated (inflated) through a hole in the bone, through a tube, or through a tube-like implant section, such as is found in cannulated bone screws. Each fastening method includes a biodegradable essentially inert atraumatic membrane. The membrane is, in part, positioned on the periosteal bone surface and abuts tissues such as tendons, ligaments, or cartilage where tissue irritation has continued to be problematic and where additional fixation means are required due to insufficient bone support.

The plates, and other fasteners used with these flexible membranes can be, in part, metallic, polymeric such as PEEK, or carbon fiber reinforced PEEK, biodegradable polymers, or bio-incorporable calcium phosphate composites.

The tensioning members can be a cable of woven or braided slow hydrolyzing biodegradable polymeric material found in disclosed the expandable device. In addition, the tensioning mechanism can be deployed in conjunction with fasteners, such as cannulated bone screws, which, themselves can apply some stability and even compression between tissues in addition to providing a rigid plate lock to provide a buttressing function. The combined use overcomes, the disadvantage of having flexible suture-type connections that are unable to resist deflection, and unable to transfer significant axial load to a plate segment, when buttressing is really needed for stability.

According to another broad aspect of the invention, the construction material of the expansion membrane is preferably a porous fabric or film that is atraumatic to perk articular gliding tendons, ligaments, cartilage and bone.

According to a further broad aspect of the invention, the invention is used in skeletal regions where other fasteners, such as bone screws, have been known to have both insufficient holding power, and/or cause tissue irritations, cartilage disruption, and even tendon, vascular, or nerve rupture.

A flexible biocompatible tensioning fastener is used secure biological materials, such as bone fragments, tissue fragments, and combinations of bone and tissue fragments. The fastener has a connector with a distal end and a proximal end, a flexible biocompatible membrane member secured to the connector's distal end. The connector member is selected from the resorbable and non-resorbable materials groups comprising, metallic cables, woven cords, fibers, strands, K-wires, polymeric materials, and composites and has means for locking the connector at the proximal end. The membrane is formed from a material that is biocompatible with one or more biological materials and is of a woven porous resorbable or degradable polymeric material having a Young's modulus in the range of 1 to 50 GPa and a tensile strength in the range of 0.1 to 20.0 Gpa. A planar fastening member that is substantially conformable to the shape the biological materials is secured to the connector proximal end. The planar fastening member can be a second membrane of a woven porous resorbable or degradable polymeric material having a Young's modulus in the range of 1 to 50 GPa and a tensile strength in the range of 0.1 to 20.0 Gpa, The planar fastening member can also be a plate having a plurality of connector receiving holes, said connector being fixed to the plate member which is secured to bone or tissue fragments, or combinations thereof. Alternatively the planar fastening member can be bone, tensioning bar or other suitable material.

The fastener can further comprise deploy tube with the connector member and folded membrane being within the deploy tube. A block tube having a distal end and a proximal end can also be used with said connector member being within the block tube and the deploy tube. The deploy tube has a distal end within the block tube positioning the first membrane proximate the block tube distal end, The deploy tube is movable relative to the block tube such that moving the deploy tube toward the distal end releases the first membrane from the deploy tube. The connector that is looped through first membrane is tied off, either through knotting or with a locking member, proximate the proximal end of the block tube.

Alternatively the can comprises an inflatable biodegradable tubular balloon having a proximal end and a distal end having an enlarged head region. The balloon can be filled with a biodegradable bone cement, a hardened bio-incorporable or biodegradable polymer, or a ceramic composite. A reinforcing rod can be used within the tubular balloon, extending substantially from the distal end to the proximal end of the tubular balloon.

The securing of the biological materials is accomplished by creating a passage having a distal end and a proximal end through the biological materials. A flexible connector having a length greater than the length of said passage is inserted through the passage and secured to a first said securing member, flexible biocompatible membrane, on the exterior of the distal end of the passage. The proximal end of the connector is locked. For insertion the membrane is and placed, along with the flexible connector, into a deployment tube. The deployment tube is inserted into the passage and the membrane and the connector deployed from the deployment tube to position the membrane external the distal end of the passage. The deployment tube is then removed and the connector locked at the external side of the proximal end, applying compressive force to the biological material. The connector is secured to a plate, or other planar member that is secured to bone.

Alternatively, the connector and membrane can be replaced with an inflatable biodegradable tubular balloon fastener which is inflated, compressing the fragments. The tubular balloon fastener has an enlarged head region at its distal end, which at least partially extends beyond the exterior of the distal end of the passage. The balloon fastener is secured at the proximal end of the passage, prior to inflation. The balloon fastener is secured to at least one fragment proximate the proximal end of the passage by a biodegradable or bio-incorporable lock plug. The balloon fastener can be filled with a biodegradable bone cement, a hardened bio-incorporable or biodegradable polymer, or a ceramic composite such as tricalcium phosphate and/or a hydroxyapatite ceramic.

### BRIEF DESCRIPTION OF THE DRAWINGS

The invention will be described in conjunction with the accompanying drawings, in which:

FIG. 1 is a side view of a one-piece fabric unit flexible member and suture, within a cutaway of a bone, employed for fixation of bone fragments to a bone, in accordance with an embodiment of the invention;

FIG. 2 illustrates a dual membrane fastener in accordance with an embodiment of the invention;

FIG. 3 shows a bone plate secured to bone fragments and bone in combination with the dual membrane fastener of FIG. 2, in accordance with an embodiment of the invention;

FIG. 4 illustrates a side view of the plate and membrane fastener of FIG. 3, in accordance with an embodiment of the invention;

FIG. 5 is an enlarged view of the system of FIG. 3 and FIG. 4 from another angle, in accordance with an embodiment of the invention;

FIG. 6 illustrates a pair of membrane fastener systems without plating, in accordance with an embodiment of the invention;

FIG. 7 is a cross sectional view of a bone, showing the suture and a pair of membranes;

FIG. 8 is a cross sectional view of a bone illustrating a pair of membranes with the suture being within a retraction block tube; in accordance with an embodiment of the invention;

FIG. 9 illustrates the suture, membrane and retraction block tube of FIG. 8 as it would be located within a bone, in accordance with an embodiment of the invention;

FIG. 10 shows a prior art dual threaded pitch differential screw.

FIG. 10A shows prior art fully tapered variable pitch cannulated screws that lock to plates.

FIG. 11 shows a deployment plunger inserting the retraction block tube along the suture that is being held by the membrane, in accordance with an embodiment of the invention;

FIG. 12 shows a membrane folded inside an insertion and block tube, in accordance with an embodiment of the invention;

FIG. 13 shows a suture attached to the folded membrane, in accordance with an embodiment of the invention;

FIG. 14 shows the structure of FIG. 13 being inserted through a bone, in accordance with an embodiment of the invention;

FIG. 15 shows the unfolded membrane, deploy plunger and a deploy tube that holds the folded device and may remain implanted, in accordance with an embodiment of the invention;

FIG. 16 is a cross-section view that shows the suture extending through the enclosing structures and looping through the membrane,

FIG. 17 illustrates an external view that corresponds to the cross-sectional view of FIG. 16, in accordance with an embodiment of the invention;

FIG. 18 corresponds to FIG. 17, showing the bones in cross-section thus exposing the deploy tub and deploy plunger, in accordance with an embodiment of the invention;

FIG. 19 shows the fastener system used in combination with a bone plate, in accordance with an embodiment of the invention;

FIG. 20 is a top view of a plate having a head region and an elongated body region with a deployment plunger and suture extending there from, in accordance with an embodiment of the invention;

FIG. 21 corresponds to FIG. 20 and shows the flexible membrane side of the fragmented bone, in accordance with an embodiment of the invention; 22 shows a flexible membrane on the distal side of the bone and a suture that passes through the deploy tube and is attached to the flexible membrane

FIG. 23 shows a side view of a biodegradable fabric plate attached to the bone and bone fragments, in accordance with an embodiment of the invention;

FIG. 24 is side view of an inflated biodegradable catheter-like balloon within a cutaway bone, in accordance with an embodiment of the invention;

FIG. 25 is a cutaway side view of the inflated biodegradable catheter-like balloon of FIG. 24 that is filled with a biodegradable material, in accordance with an embodiment of the invention;

FIG. 26 is a further cutaway of the system of FIGS. 24 and 25, in accordance with an embodiment of the invention;

FIG. 27 is a front view of the fastening system fully employed, in accordance with an embodiment of the invention;

FIG. 28 is a cutaway of a balloon that can be pre-sized and pre-shaped such that upon inflation compression of the fragments occurs, in accordance with an embodiment of the invention;

### DETAILED DESCRIPTION

The invention relates to a fastener system for internal stabilization of a fractured bone, and more particularly to atraumatic fasteners having flexible membranes and tensioning means which can be used in conjunction with one or more plates, other fastener types of other materials, including locked cannulated screws-plate and to method of securing bone fragments which includes securing or capturing a fragment, at least in part, with an atraumatic foldable and/or expandable device.

The present inventions satisfy the goals of improved attachment strength and control of fracture fragments in regions where traditional screws and wires are ineffective due to the lack of sufficient good quality bone or where the presence of metallic objects would irritate or would endanger surrounding soft tissue function by achieving fixation using a relatively low profile biodegradable inflatable biodegradable fastening mechanisms.

Furthermore, operative trauma to the critical soft tissues which needs to be avoided is minimized by the deployment of the fastener to the critical surface from the opposing bone surface or from within the bone surface. Deploying a folded device or expanding a device at the tip to prevents retraction through the bone. In the case of an inflatable balloon expansion within the cancellous region of the bone compresses the calcium-containing soft cancellous bone surrounding the tip creating a secondary attachment of the fastener.

Another aspect of this invention relates to improvements in the surgical treatment of bone conditions of the human and other animal bone systems and, more particularly, to an inflatable balloon-like device for use in treating such bone conditions.

The disclosed system incorporates one or all of the following elements for internal stabilization of fractured/fragmented bones.

Folded expandable devices (folded membranes) with tensioning members for use in surgical protocol relating to fixation of bone fragments to bone, tissue to tissue or bone and tissue fragments to bone. In one embodiment, fixation holes in the membrane plate enable use with sutures, in another embodiment; one or more integral tensioning members are pre-assembled to the membrane. In addition the disclosed system can be used to facilitate disc repair by attaching the flexible member to the superior and inferior endplate to plug or block the “rent” in a torn disc to prevent further extrusion of nucleus post partial neucleotomy.

The folded device may be one of several relatively flexible structures or materials such as biocompatible polymer, expandable Nitinol (NiTi alloy) structures. However, the primary focus of the disclosed system is to use biodegradable materials in components that grip bone and tissues in positions that are near moving structure such as tendons or ligaments. In addition it is preferred the material retain its strength for about 12 to 24 months, minimum. In small bones, the folded membrane typically has only one tensioning means (for example, one looped suture, or one integral cord or braid).

The membrane will become substantially flat upon deployment and can be a semi-flexible membrane of any generic shape, but preferably a round, or polygonal (e.g. triangular, square, rectangular, octagonal et cetera), onto a ligament/tendon/bone facing surface. The membrane preferably contains a resorbable or degradable polymeric material having a Young's modulus in the range of 1 to 50 GPa and a tensile strength in the range of 0.1 to 20.0 Gpa. The membrane can be a film or, preferably, a woven porous structure.

Flexible membrane plates for use in surgical protocol relating to fixation of bone fragments to bone, or bone and tissue fragments to bone. In one embodiment, multiple reinforced fixation holes in the membrane plate allow use with multiple sutures, screws, staples, tissue anchors or other bone attachment means, in another, one or more integral tensioning members are pre-assembled to the plate.

These plates by be folded for expansion and deployment in openings as per (A) or, but due to their size and the need for multiple fixation points, they will frequently be inserted using a small incision to facilitate secondary tack down of the plate to bone using secondary bioincorporable fasteners or secondary sutures and/or suture anchors, These flexible plates can not resist bending or provide buttressing (unless used with buttressing plates and cannulated screws), However, they can resist tension, and, as such, can be installed in a manner to resist gapping of a fracture gap under tension (tension banding).

Inflatable bio-incorporable balloon device for use in surgical protocol relating to fixation of bone fragments to bone, or bone and tissue fragments to bone. Preferably the balloon is preshaped and comprised of a biodegradable or bioincorporable polymer or like material.

Combined use of (A), (B), or (C) (above) with a plate, or plates and screws, or plates and cannulated screws, or more specifically with plates and locked cannulated screws. The combination provides greater fragment control and stability that is desirable in certain fracture patterns.

The fasteners can be secured to, or deployed through many traditional bone repair devices such as intramedullary nails, cannulated screws, or traditional bone plates and washers.

Although the membranes illustrated herein are multiple independent buttons, fabric flexible membranes can be elongated to have multiple attachment points. The elongation of the membranes to form plates will, as some point depending upon size and thickness, require a separate stab incision for installation as they will not be small enough to deploy through the bone. Whether in independent buttons or elongated membranes, the disclosed system provides flexible suture plates of bio-inert bio-degradable materials which allow better capture of per-articular fragments than internal fasteners such as bone screws, yet are atraumatic to the overriding tendons, (See histologies for Melon CNC spacer.)

The membrane material used herein must be non-cytotoxic, and the foreign body reaction minor. In this application it is preferable to have a slowly degrading material which does not swell extensively during degradation or result in extensive fibrous scar formation or a late inflammatory response as has been observed with the use of slowly degrading highly crystalline poly (L-lactide). It is also preferable for it to be somewhat flexible and strong. It must be easy to deploy, and yet strong enough to the filler and tissue fragments in place and distribute stresses over the membrane surface after deployment.

Flexible fabric membranes used in fracture repair can be seeded with adult stem cells, cartilage cells, fibroblasts, bone marrow, other types progenitor cells or substances such as bone morphogenic proteins, morselized cancellous bone or bone marrow aspirate to help induce the desired repair (callus formation, bone formation or cartilage formation). Mesenchymal stem cells are progenitor cells that have the capacity to differentiate into more specialized daughter cells. Types of progenitor cells include hematopoietic and embryonic stem cells. Mesenchymal stem cells reside in bone marrow, fat, skin, and muscle and around blood vessels (as pericytes), and have the capacity to differentiate into bone, cartilage, muscle, marrow, and other connective-tissue cells. To deliver the mesenchymal stem cells to the site of injury, cultured cells can be placed in a degradable delivery system (typically a scaffold) onto which the cells can adhere. The delivery matrix and attached cells are then placed at the site of injury, where the cells can carry out their synthetic functions while adhering to a structurally competent matrix.

Similarly, materials or compounds to prevent adhesions can be added when appropriate. Many juxta-articular injuries are near a tendon or may even lacerate the tendon. Many injuries also disrupt the nutritional systems that feed the tissues sustain the repair effort. Operative intervention can interfere with the biology of healing. It has been observed that adhesions form in proportion to the degree of crushing and manipulation of the tendon tissue during surgery. The effects of surgical trauma are superimposed on those of the original injury and contribute to a biologic environment that is favorable for scarring.

The suture, or connector, materials used can be any biocompatible material and can include resorbable and non-resorable materials, for example polymeric materials and composites as well as metallic cable.

The filler material is a bone replacement material that preferably completely degrades, but initially has properties equivalent to or greater than bone, but then successively decreases in resistance to force, as a result of which the endogenous bone transformation processes (remodeling) are stimulated and hence more rapid osteoneogenesis and hence also active resorption of the bone replacement material is introduced. However, preferably, the regional strength of the fastener is maintained either via reduced resorption or increased ossification for a period of about 1 to 2 years. The material must be workable for long period of time at room temperature, must be able to form may be formed at low temperatures, is readily formable and/or injectable, and yet can harden to high strength upon further reaction. To assure good fixation, the material cannot shrink as it consolidates. A material with low-grade expandable properties is also desirable.

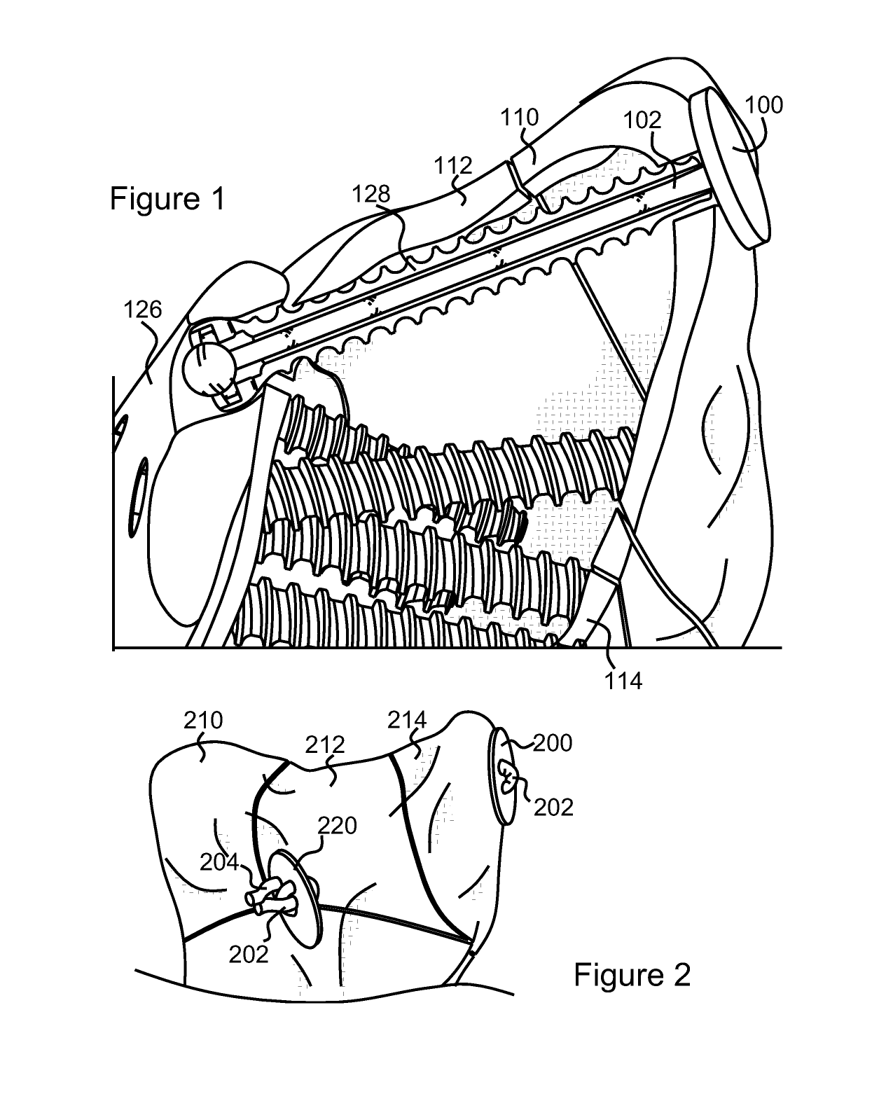

One embodiment of the disclosed fastener is illustrated in FIG. 1, a one piece fabric unit flexible membrane member 100 and suture 102 are employed for fixation of bone fragments 110 and 112 to bone 114, The suture 102, in this embodiment, has been passed through a threaded fastener 128 that has been locked into a plate 126 as known in the art. It should be noted that the lock illustrated herein could be replaced with a crimped collar when a metallic cable is used.

FIG. 2 shows the use of dual membranes, 200 and 220, used to attach bone fragments 214 and 212 to one another. The suture 202 is looped through the first membrane 200, passed through a hole drilled in the bone fragment 214, as described hereinafter, and secured at the ends 204 with a second membrane 220 in an adjoining fragment 212. This process would be repeated until fragments 210, 212 and 214 are joined to one another and to the bone 216.

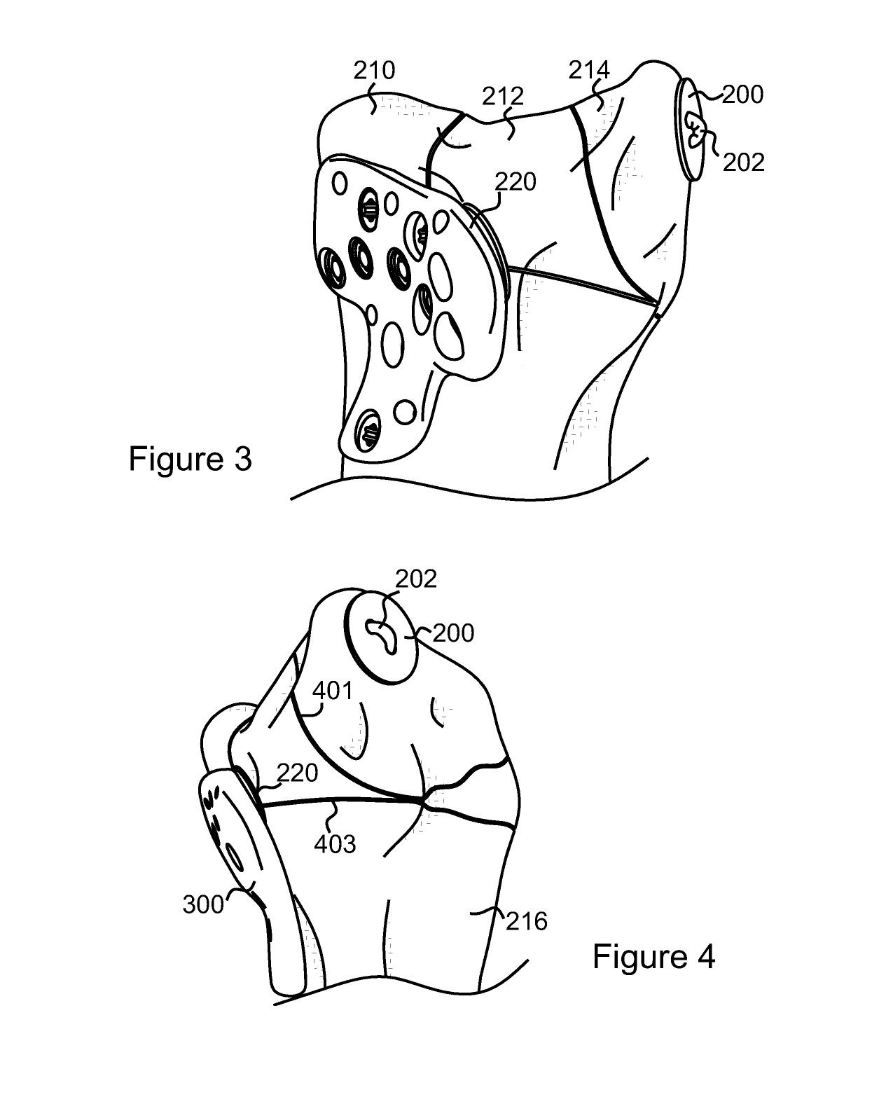

In FIG. 3 the bone plate 300 has been placed over the membrane 220 and secured to bone fragments 210 and 212 and bone 216. As noted above, the membrane 220, as well as any additional membrane/suture combinations required, is used to attach the fragments 214 and 212 to one another through the use of suture 202, to be subsequently secured to the bone 216 through use of the bone plate 300.

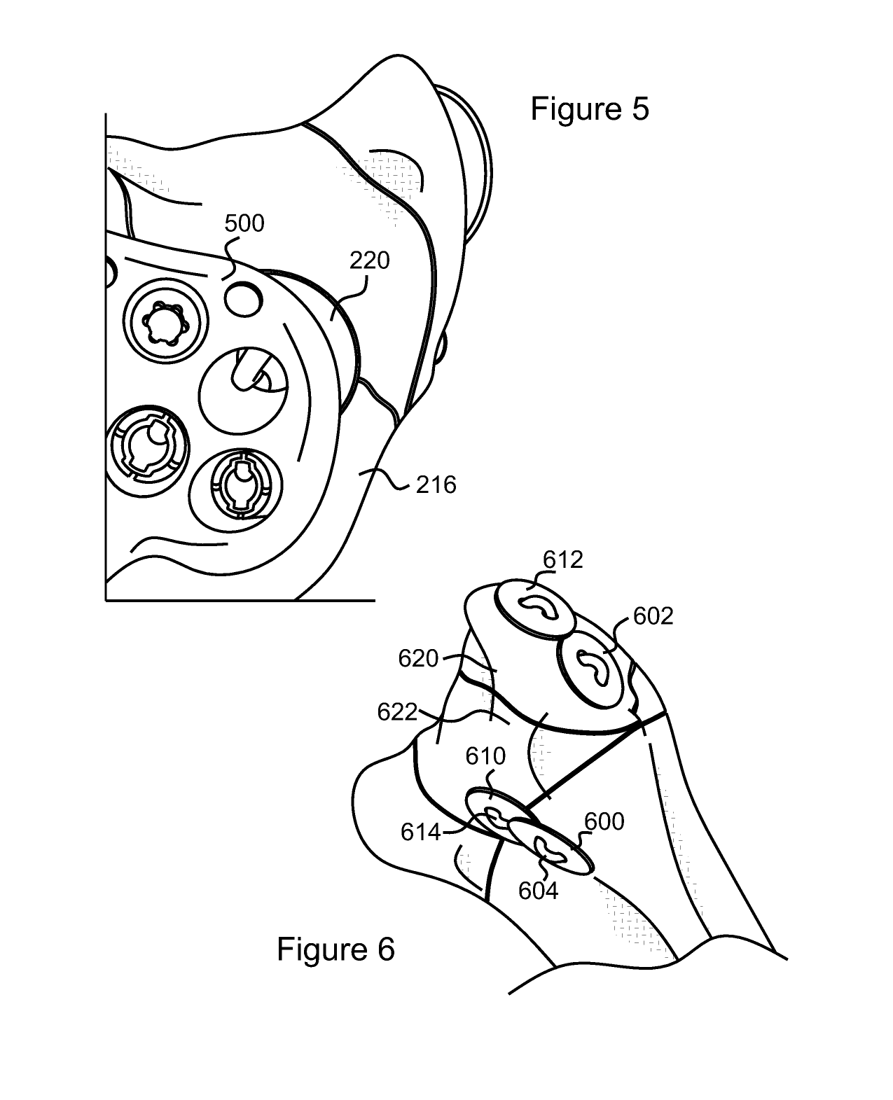

The bone of FIG. 3 has been rotated in FIG. 4 to show the bone 216, membranes 220 and 200 and suture 202 from another angle showing fracture lines 401 and 403. The enlarge view of FIG. 5 shows the system of FIG. 3 and FIG. 4 from another angle, specifically illustrating the interaction between the plate 300 and the membrane 220. Additionally, one or more screws 500 are used to secure the bone plate 300 to the bone 216.

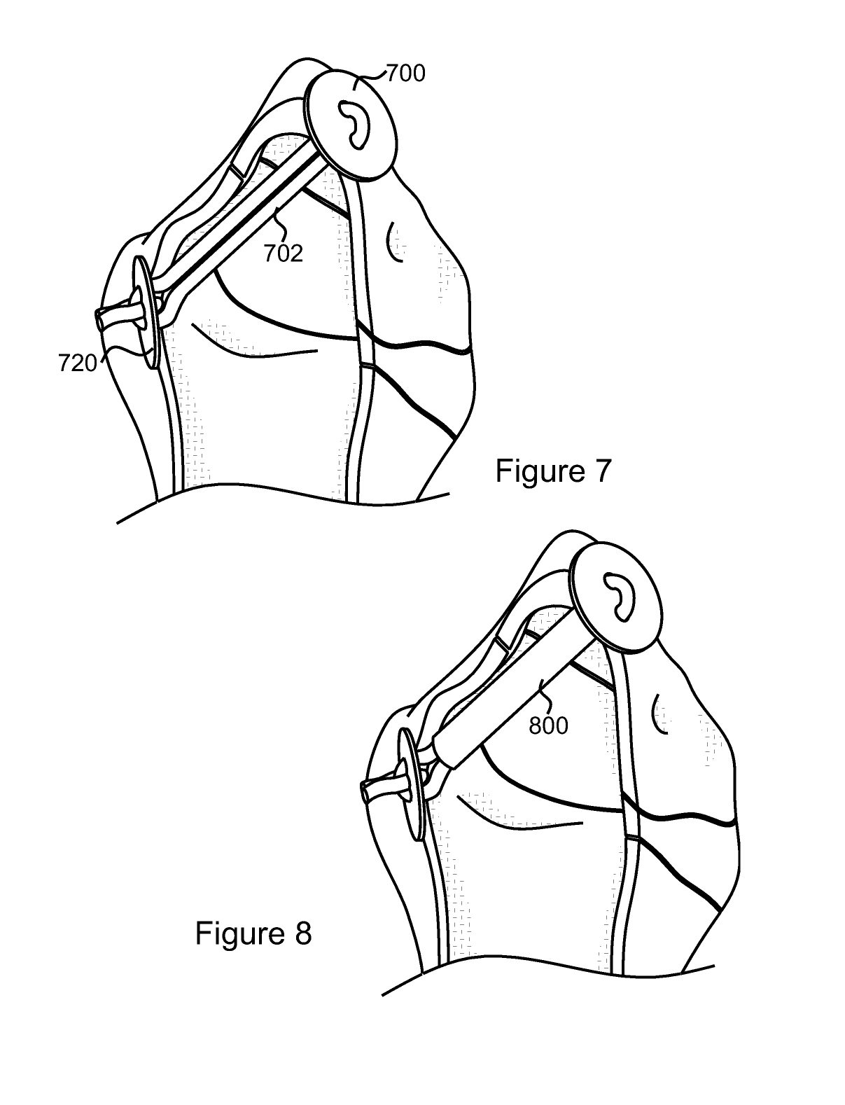

FIG. 6 illustrates a pair of membranes 600 and 602 and 610 and 612, securing bone fractures without a bone plate. The membranes 600 and 602 are attached through suture 604 and membranes 610 and 612 are attached to one another through suture 614. In this example, fragments 620 and 622 are secured to one another through membranes 610 and 612 and then secured to the bone 630 through membranes 602 and 600. As noted heretofore, the membranes and sutures can be used alone, as illustrated in this figure, or in conjunction with plates and other fixation devices.

FIG. 7 is a cross sectional view through a bone, showing the suture 702 and pair of membranes 700 and 720.

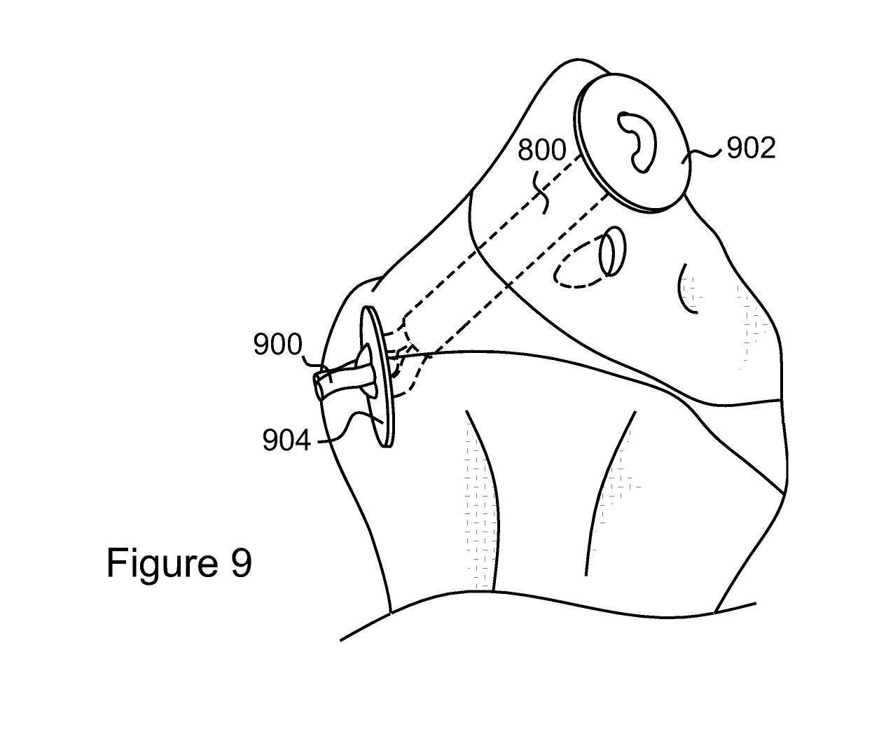

In FIGS. 8 and 9 a biodegradable retraction block tube 800 is interposed between two membranes 902 and 904. The suture 906 is looped through the membrane 902, passes through the block tube 800 with a knotted suture lock 900 used to lock the system in place.

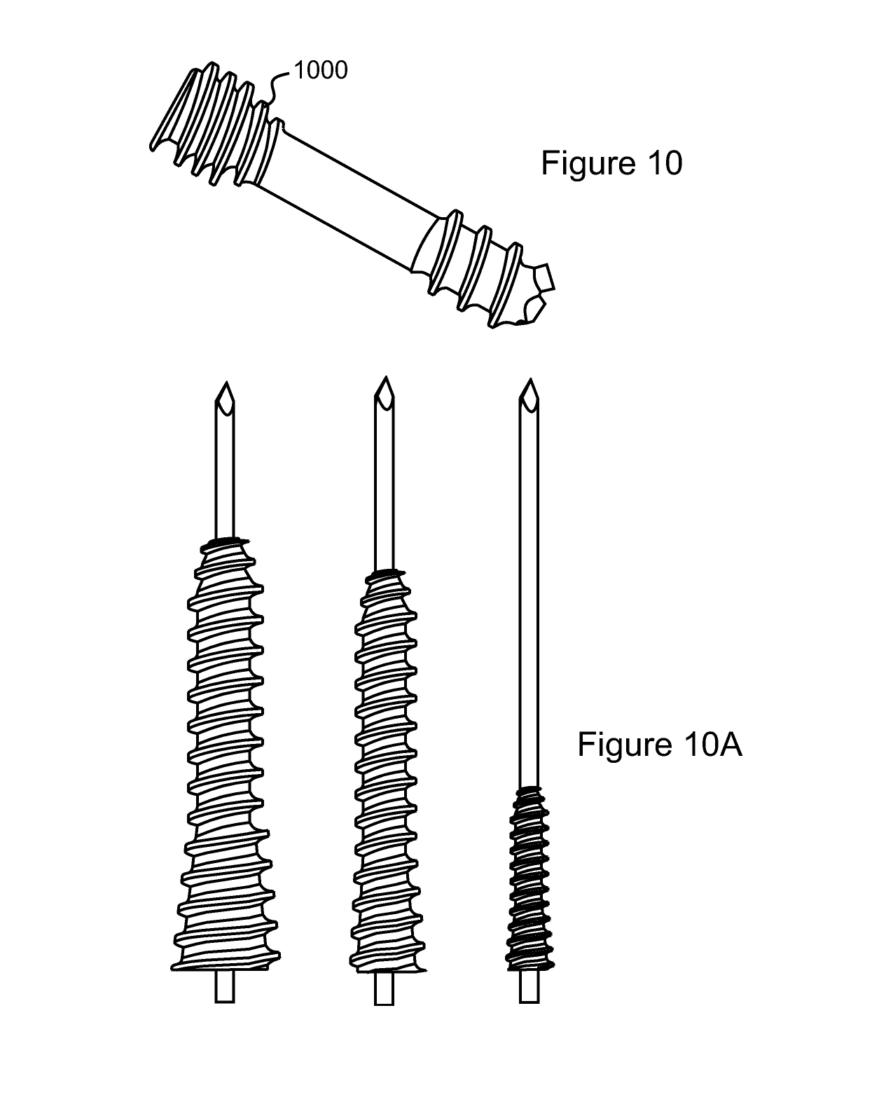

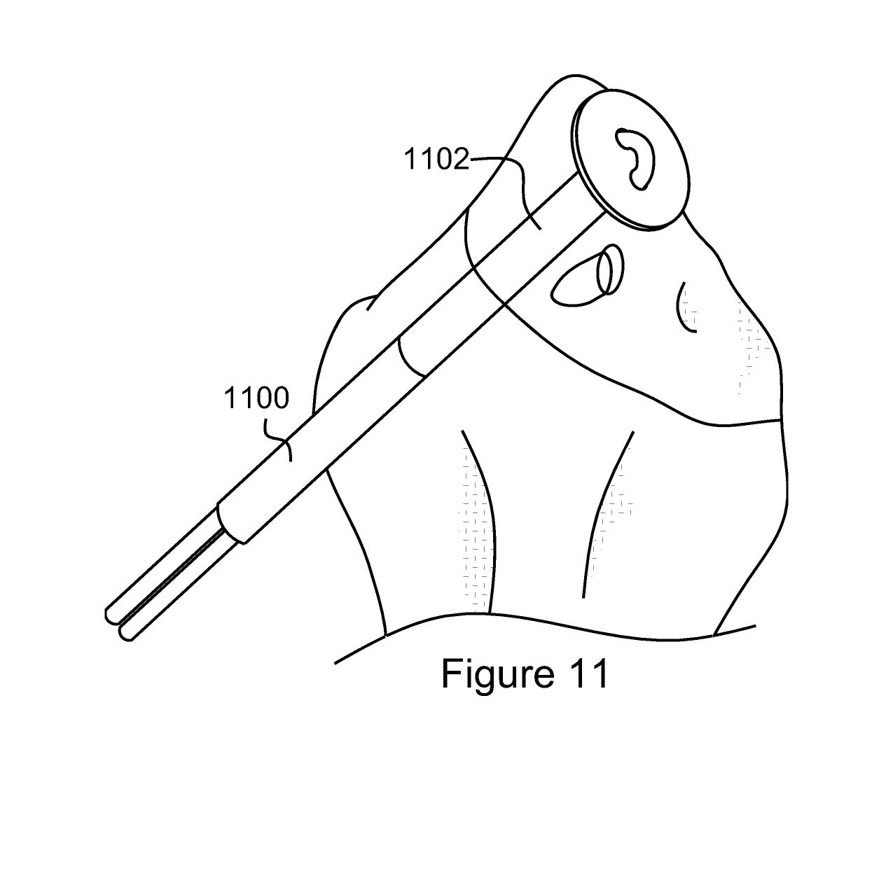

Cannulated screws that lock to plates or dual threaded pitch differential screws 1000, such as illustrated in FIG. 10, can be pre-loaded with the deployable membrane 200 and used alone or in conjunction with plates. In addition fully tapered variable pitch screws cannulated screws, as illustrated in FIG. 10A, can also be pre-loaded with the disclosed deployable membrane 200 and used alone or in conjunction with plates. The membrane 200 is preloaded into cannulation of the screws prior to or after screw insertion, depending on cannulation size relative to membrane geometry. After the cannulated screw is inserted, the membrane 200 with cord or suture attached is deployed, retraction of the membrane is blocked, and the near-side secured in one of several fashions as outlined hereinafter.

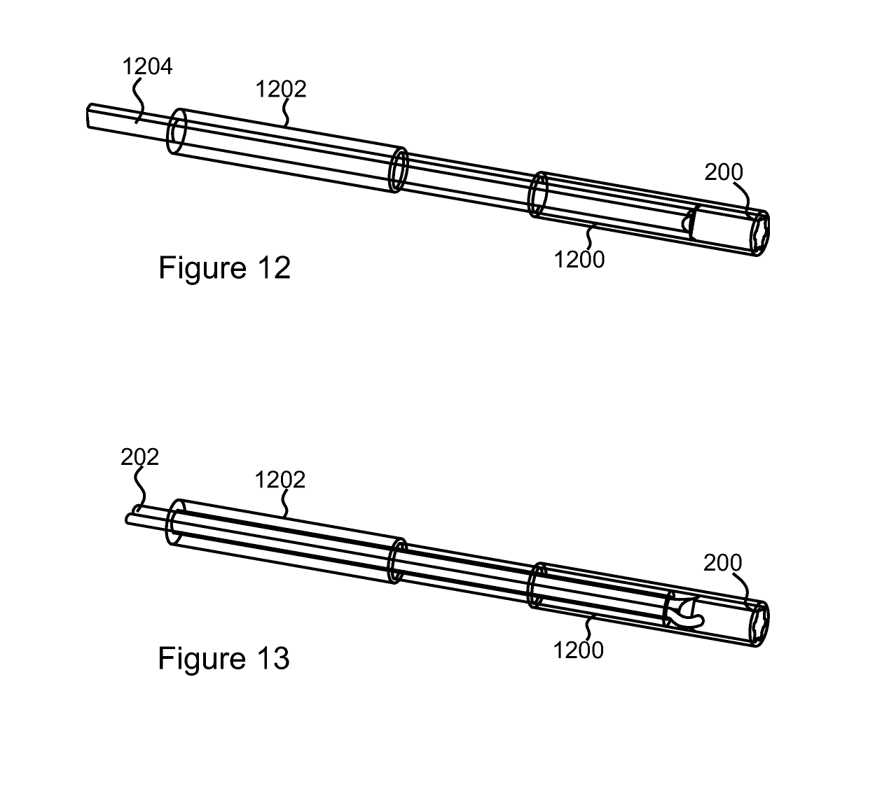

The retraction block deployment tube 1102 and deployment plunger 1100 are available in 1 mm increments and would be dimensioned as known in the art. Break away rings can be incorporated on the locking tube 1200, of FIG. 12 to facilitate shipping of one length of blocking sleeve for the device.

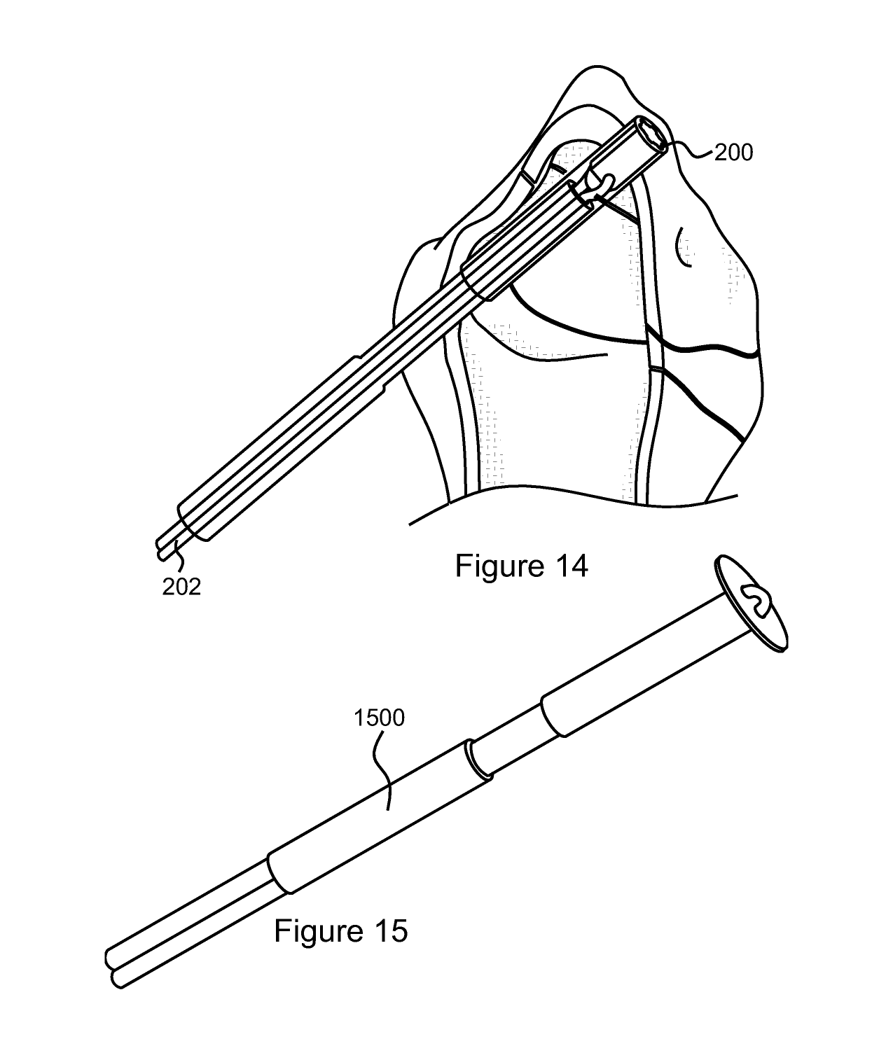

FIGS. 12 and 13 shows the membrane 200 folded inside an insertion and block tube 1200. Adjacent to the insertion and block tube 1200 is the deploy tube 1202. The suture, or woven cord, 1204 is threaded through the folded membrane 200 with both open ends threaded through the insertion and block tube 1200 and deploy tube 1202. In order to enable securing of the suture 1204, the open ends of the suture 1204 extend beyond the deploy tube 1202, the distance of which is dependent upon the securing means and will be known to those skilled in the art. In this illustration the membrane 200 is shown folded one direction, however this can vary, depending on properties and size of a membrane. In some applications the fold will need to be in the opposite direction than shown FIG. 12 to assure “flat” deployment against bone when the membrane's suture, or cord, 1204 is tensioned and locked in place.

In FIG. 14 the insertion and block tube 1200, carrying the membrane 200, and deploy tube 1202 are illustrated being inserted through a bone.

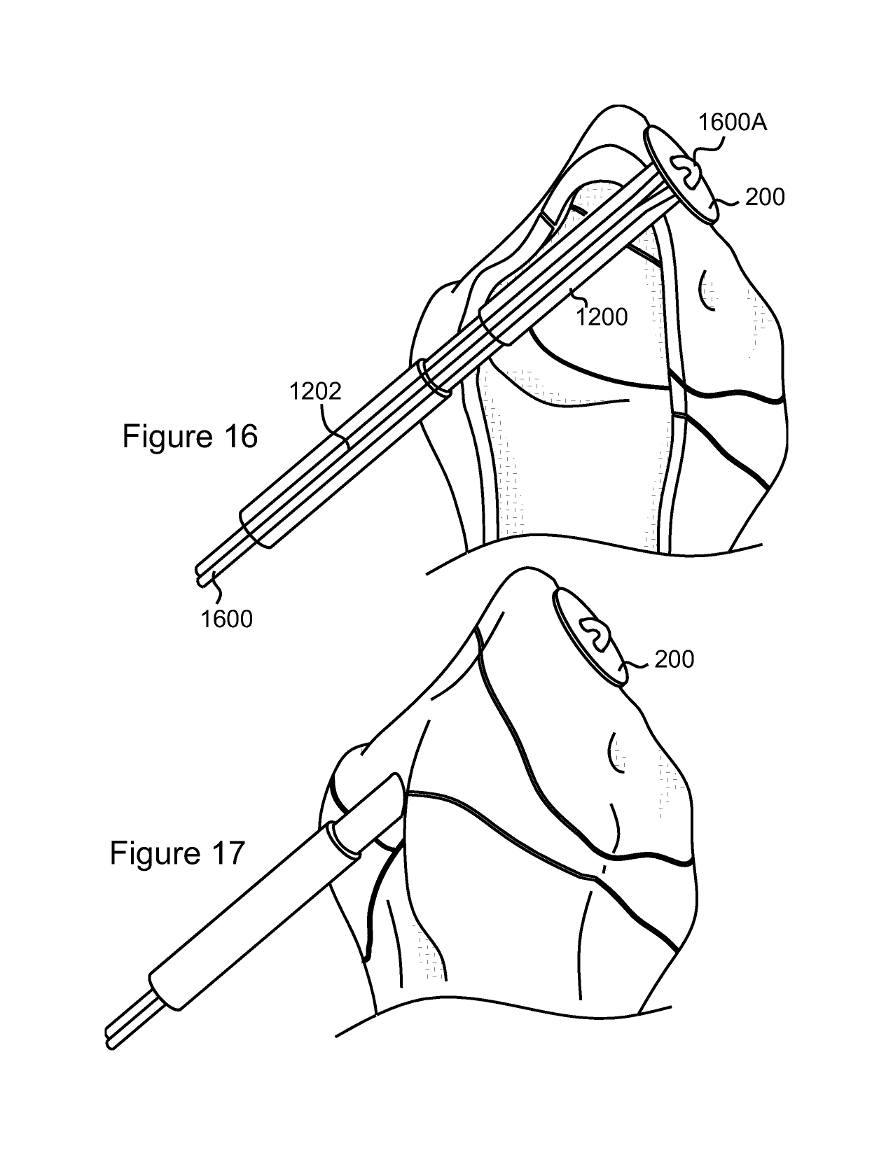

In FIG. 15 the folded membrane 200 of FIGS. 12-14 has been deployed by pushing the deploy tube 1202 and forcing the insertion and block tube 1200 and membrane 200 out of the deploy tube 1202. This releases the membrane 200 which is held in place by the deploy tube 1202. The deploy tube 1202 can be bio compatible, using materials known in the art, and remain implanted. It should be noted that the deploy tube 1202 in some applications the will be replaced with a cannulated screw or other cannulated device.

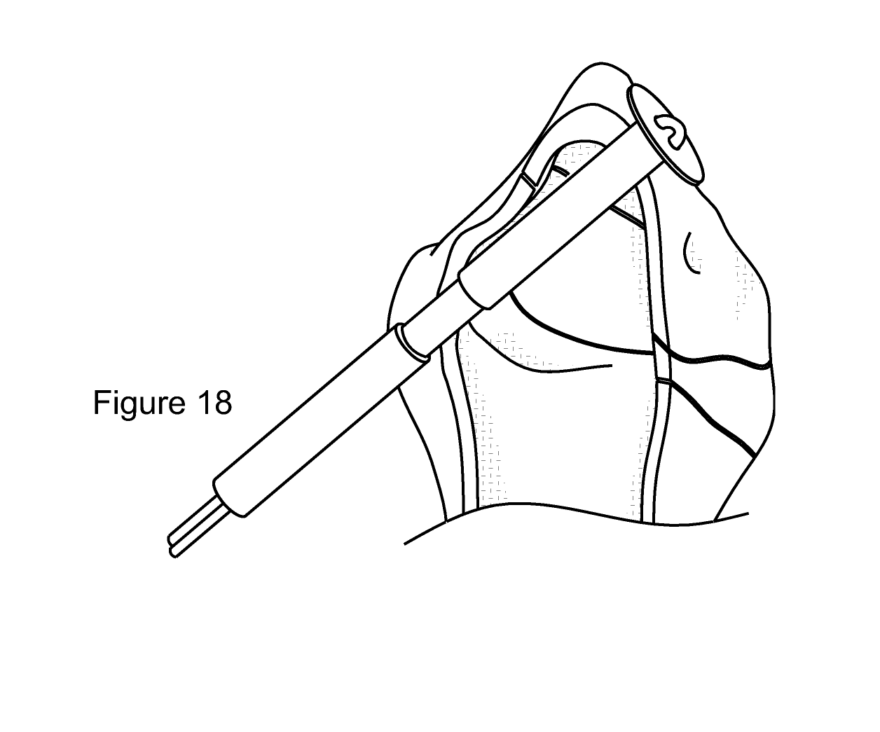

The deployment of the membrane 200 is illustrated in FIG. 16 as it would be when inserted through a bone. This cross-sectional view shows the suture 1600 extending through the deploy tube 1202 and insertion and block tube 1200 and looping through the membrane 200, at 1600A.

FIG. 17 shows the external view of the insertion process illustrated in the cross-sectional view of FIG. 16. In FIG. 18 corresponds to FIG. 17, except that it shows the bones in cross-section thus exposing the deploy tube 1202 and the insertion and block tube 1200.

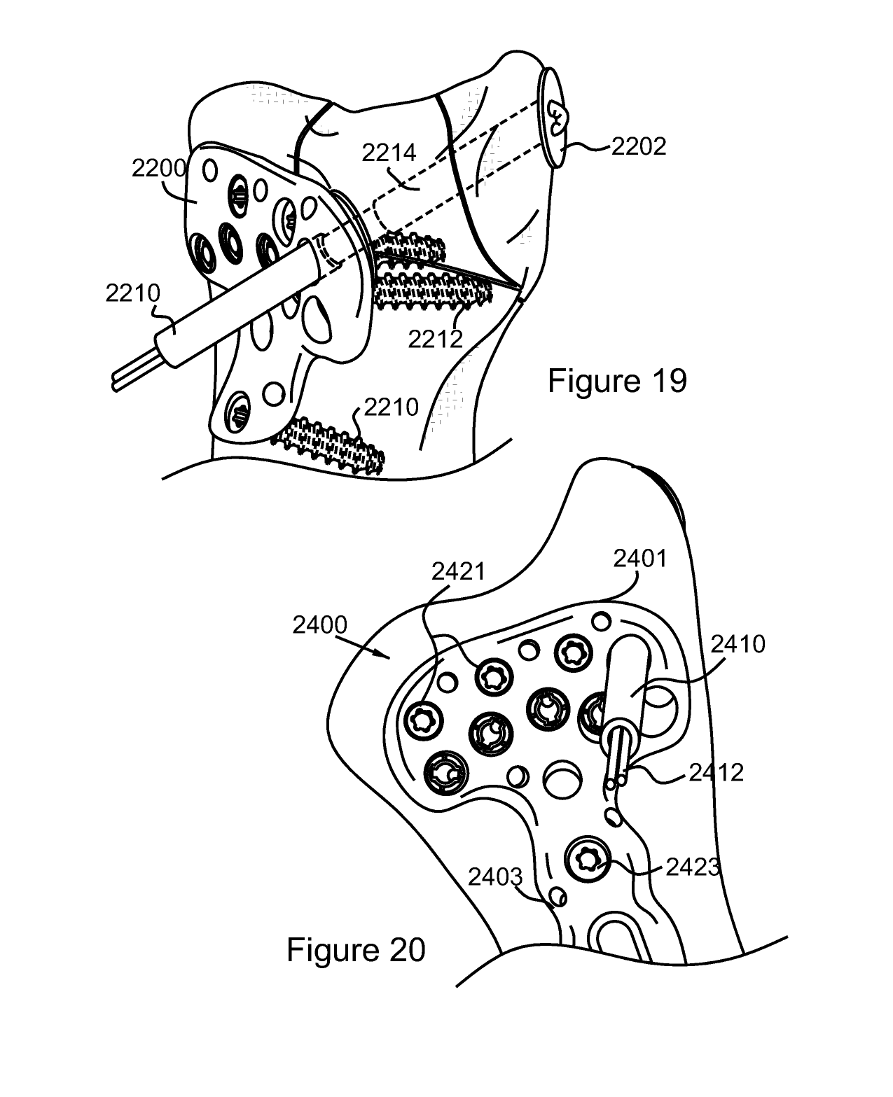

FIG. 19 shows the elements of the disclosed fastener system used in combination with a bone plate 2200. The fastener system of FIG. 19 includes the flexible membrane 2202, the insertion and block tube 2214, the deploy tube 2210. Also illustrated are the screws 2212 extending through the elongated body of the plate 2200, and screws 2212 that extend through holes in the head of the plate 2200, into bone fragments.

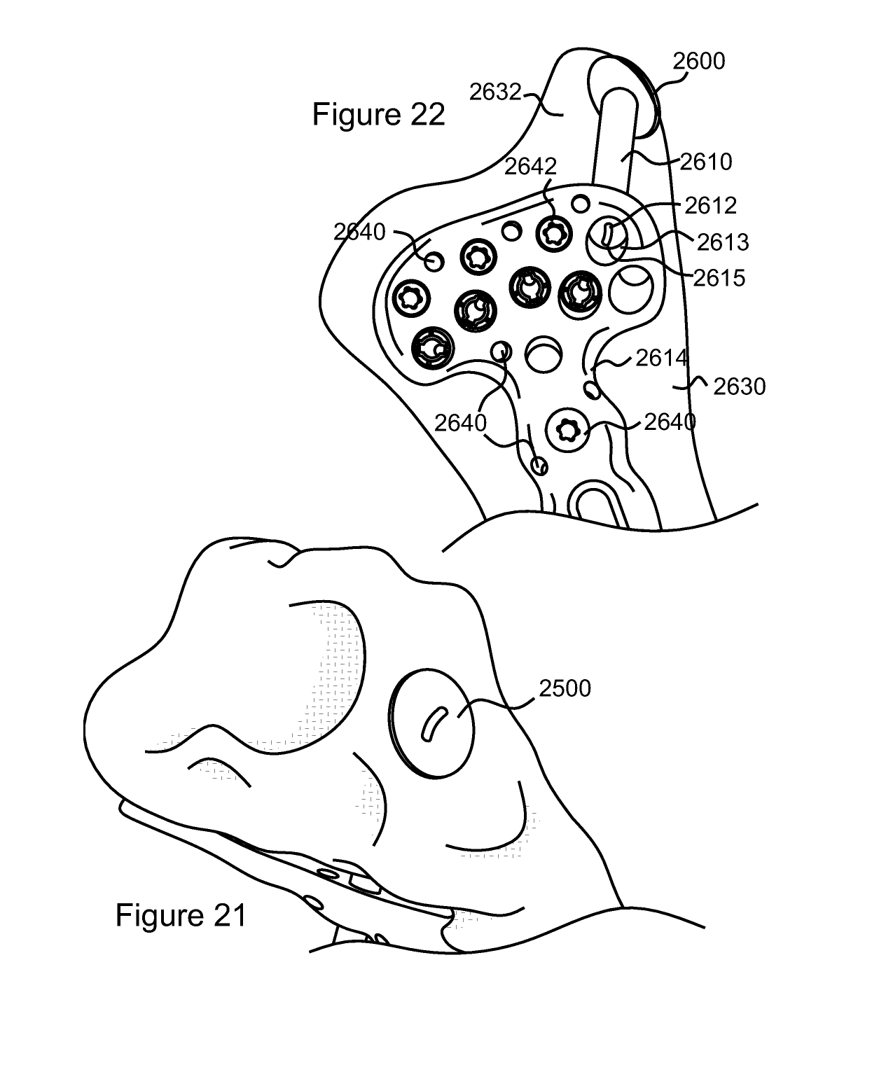

FIG. 20 is a top view of a bone plate indicated generally as 2400, and having a head region 2401 and an elongated body region 2403. A plurality of screws 2421 or other fasteners are employed in the head region 2401 to secure the plate 2400 to bone fragments 2430. The flexible membrane fastener system having a deploy plunger 2410 and a suture 2412 are employed through one of the holes in the head 2401 to secure the fragments. One or more screws or other fasteners 2423, or mixtures of fasteners can be employed to secure the elongated body region 2403 to the bone 2432. The opposing side of the system illustrated in The opposing side of the bone illustrated in FIG. 20 is illustrated in FIG. 21 showing the flexible membrane 2500 side of the fragmented bone.

As seen in FIG. 22 the flexible membrane 2600 is on the distal side of the bone 2630. A suture 2612, which has been attached to the flexible membrane 2600, passes through the insertion and block tube 2610, as described above. The suture attaches to the plate 2614 through a pair of holes 2615 in the recess area 2613. Four holes can be provided to accommodate a pair of sutures. The flexible membrane 2600, insertion and block tube 2610, and suture 2612 combination serve to compress the bone fragments on either side of the fracture 2632. The plate 2614 is secured to the non-fragmented bone and adjacent bone fragments by a plurality of screws 2642, or equivalent fastening device or devices, in addition to the flexible membrane system. At least one screw or fastener 2642 is provided in the body part of the plate 2614 for securing the plate 2614 to the primary length of bone 2630. The plate 2614 can be provided with a plurality of holes 2640 to enable us of other securing members, such as for example, k-wires, bolts, or screws.

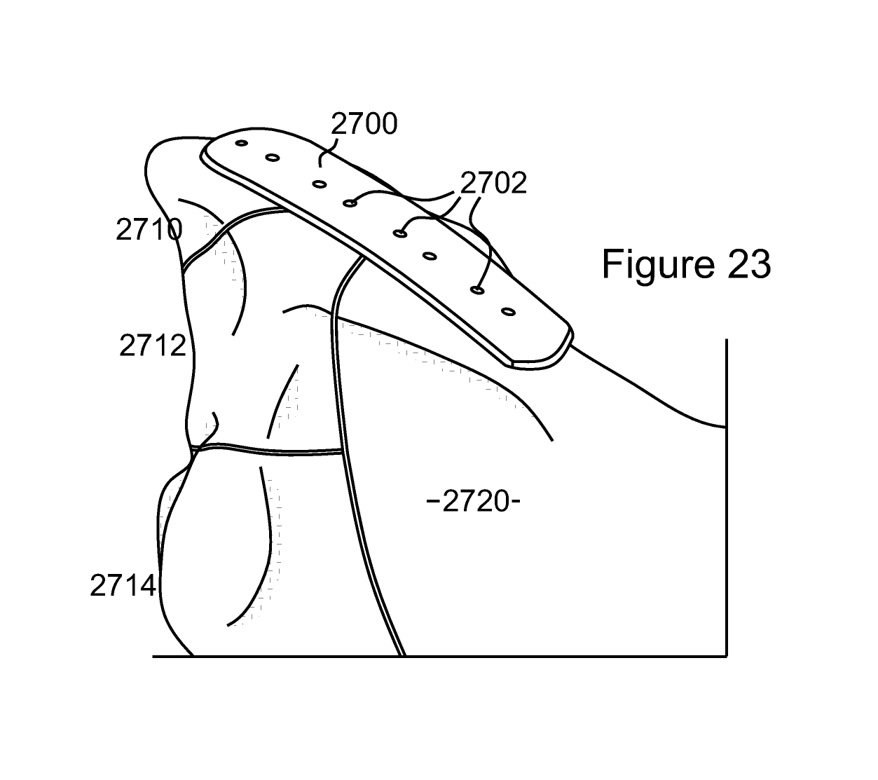

In another embodiment of the invention, as illustrated in FIG. 23, a biodegradable fabric plate 2700 can be attached to the bone 2720 and bone fragments such as 2710, 2712, and 2714, via sutures, not shown. A plurality of holes 2702 are provided in the plate 2700 for sutures, k-wires, or the like. A targeting guide can be provided to link sutures to plate when or if desired.

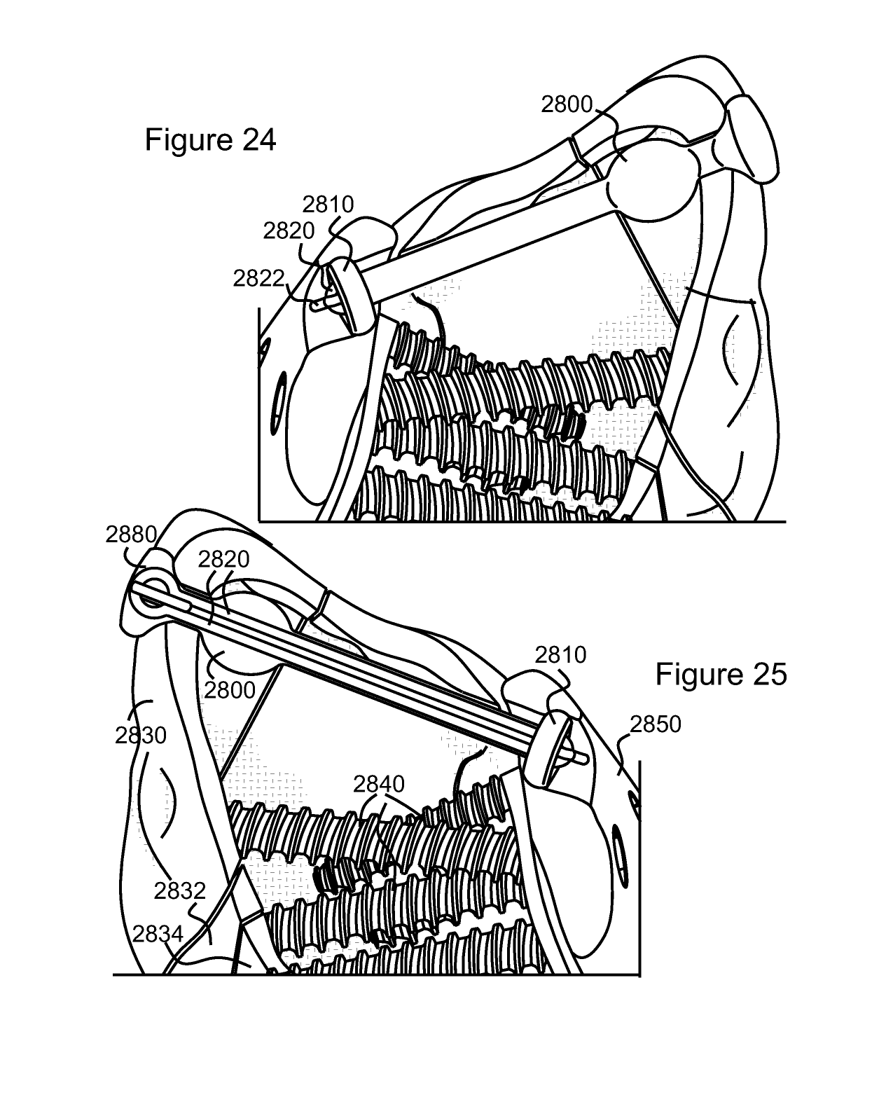

An alternate fastener system is shown in FIG. 24 and FIG. 25. In this system, an inflated biodegradable catheter-like balloon 2800, is filled with a biodegradable bone cement or similar hardened bio-incorporable or biodegradable polymer, ceramic composite (typically containing tricalcium phosphate and/or hydroxyapatite ceramics), or the like. The system includes a biodegradable or bioincorporable lock plug 2810 and can also include suture ties 2820. A knotted suture lock 2822 can be provided to secure the suture ties 2820. The full fastening system, inclusive of the balloon 2800, suture ties 2820, plate 2850, and screws 2840, can be employed to compress the bone fragments 2830, 2832 and 2834.

In one embodiment, the balloons 2800 are pre-sized and pre-shaped such that, upon inflation, compression of the fragments occurs, as shown in FIG. 25. eliminating the need for a secondary tensioning step and a secondary locking step. To facilitate installation, a reinforcement 2820 will typically be pre-installed into the deflated balloon 2800. The reinforcement 2820 will be used in the positioning of the balloon implant and can have protrusions or undercuts or other surface roughening to facilitate incorporation with the expansion filler material. In this embodiment the balloon 2800 is secured to the exterior of the bone through use of the enlarged head 2880. A plate 2850 has been affixed to the bone with screws 2840 to secure the fragments 2830, 2832 and 3834.

The thickness of the balloon 2800 wall is typically in the range of 2/1000ths to 25/1000ths of an inch, or other thicknesses that can withstand pressures of up to 250-400 psi. The filler instrumentation will control fill pressure or fill volume, and this fill pressure will be adjusted to match the properties of the biopolymer used for the balloon material.

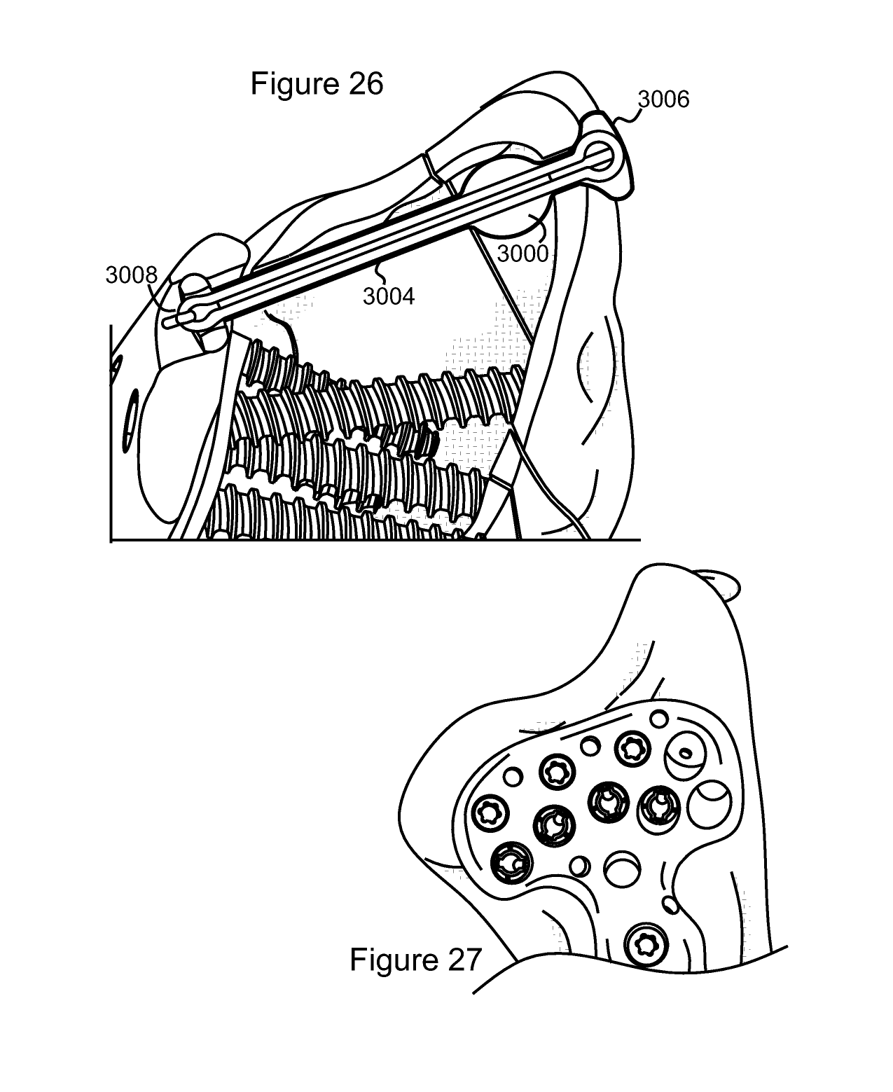

In the embodiment illustrated in FIG. 26, the reinforcement can be a length of tube 3004 as illustrated, extending within the expandable balloon 3000. The tube 3004 can any rigid or semi rigid biocompatible material of the same material as the balloon 3000, or other, at which point the enclosed length of tube 3004 provides an interior lumen passing within the expandable structure in a manner previously disclosed and shown in FIG. 15. The lumen can accommodate the passage of a stiffening member or stylet made, e.g., from Nitinol, stainless steel, molded plastic or other material known in the medical arts, which serves to keep the structure in the desired straightened condition during passage through an associated guide sheath toward the targeted body region. The tube 3004 can secured via knotting or other means known in the art at the distal and proximal ends 3006 and 3008. Alternatively at the distal end 3006 the suture can be replaced with a biodegradable rod with threaded end to accept a biodegradable threaded washer. The proximal end 3008 the end of the tube 3004 can have threads to secure the proximal end 2008 to a plate.

The tube 3004 can have a preformed memory, for example to normally bend the distal region of the stylet. The memory is overcome to straighten the stylet when confined within the guide sheath. However, as the structure and stylet advance free of the guide sheath and pass into the targeted region, the preformed memory bends the distal stylet region. The bend of the distal stylet region bends the tube and thereby shifts the axis of the attached expandable structure relative to the axis of the access path, that is, the guide sheath. The prebent stylet, positioned within the interior of the structure, further aids in altering the geometry of the structure in accordance with the orientation desired when the structure is deployed for use in the targeted region. Attention is invited to FIGS. 22 and 26 of U.S. Pat. No. 5,972,015.

Once in position the balloon 3000 can be pressurized with an appropriate material such as a bioincorparable, biodegradeable polymer or bone cement, that polymerizes and hardens within the biodegradable elastomeric balloon.

Either the tip of the balloon marker or the insertion tube can be marked with a radiopaque material allowing the tip position to be tracked using fluoroscopic techniques. Additionally, the balloon can be prepackaged in a tube to facilitate easy passage, and the tube can be part of a surgical tool, simple packaging material, or part of another implant such as a screw or peg which is seated in the tissue, and which can also be affixed to or through a plate.

The block component of the appropriate length can be pre-packaged with the expandable membrane or balloon to facilitate use. When the expandable device is a pre-shaped balloon, deployment involves sealing and inflation of the balloon using a biocompatible and biodegradable or bio-incorporable injectable polymer or ceramic-polymer composite cement which, when cured (hardened) creates the expansion attachment. This also blocks the opening to prevent fastener retraction.

In addition to, or instead of a tensioning means as required in the membrane style invention, the balloon can be pre-shaped and sized to complete fastening and tensioning in one step as seen, for example, in FIG. 24. The shaping can allow for asymmetry and can be flat or the lower profile that is depicted in FIG. 24. The device can be used alone, or in combination with other fasteners such as plate or bone screws.

The biodegradable catheter-like balloon fastening systems disclosed herein can be pre-packaged in a deployment tube, (not shown) as described above. The procedure for use of the balloon system includes step 1—deploy, step 2, remove tube, seal, and fill, step 3—hardening of the filler material, and step 4—tense and knot to lock, or lock using an alternate device such as clamps, or a knotless lock device, FIG. 27 shows the fastening systems described above fully employed.

For any size screw, only a few device lengths are required when using suture tensioning and a secondary suture lock step, but multiple lengths are required for pre-shaped balloons capable of fixation of both ends and tensioning in one step.

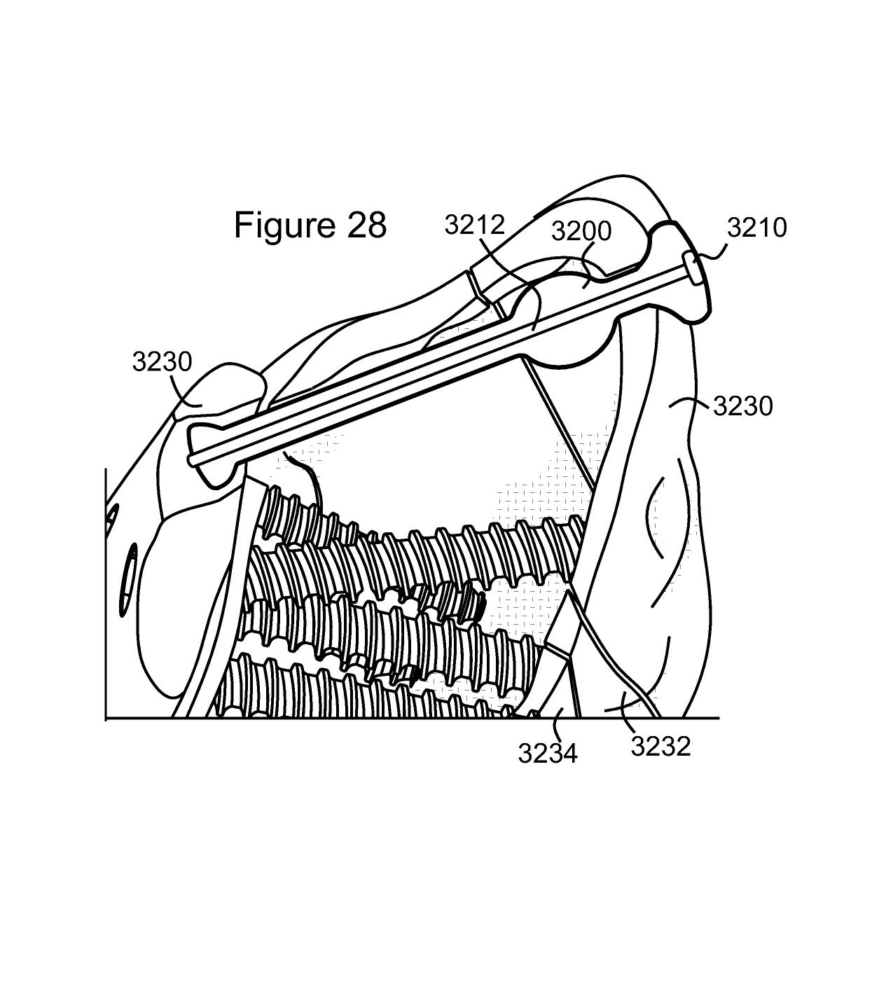

FIG. 28 shows an alternate system in which a biodegradable reinforcing rod 3212 is employed to reinforce and facilitate installations. The tip 3210 of the rod 3212 can be radio-opaque to provide for visualization of the location prior to inflation of the balloon structure 3200. The balloon structure 3200 is filled and sealed using a disposable caulking type mechanism to fill a specific amount or to a specific pressure. This is carried out in a manner similar to a measured syringe with or without a hypodermic needle as used in some 1 and 2 part epoxy systems. The seal in one instance can be via balloon twist or crimp and hold with instrumentation to seal until the filler material hardens. In another embodiment, a separate clip can be used. In another, a suture-like tie can be used. The bone fragments 3230, 3232, and 3234 are compressed together and to the plate 3230, if used, during the inflation process. With proper reduction, the fill process captures a fragment or fragments, and pulls fragments together and toward the plate, if used. The filler material for the balloon can be materials such as magnesium ammonium phosphate, or calcium phosphate cements. The reinforcing rod is preferably biodegradable and/or bio-incorporable.

Other fillers for reinforcing the cancellous bone can be methylmethacrylic or bio-incorporable Magnesium Ammonium Phosphate cements or other calcium phosphate bone cements (typically containing tricalcium phosphate and/or hydroxyapatite ceramics, or even fibrin-based or collagen-based bone glues. Examples can be seen in, Kyphon patents, particularly, U.S. Pat. No. 7,153,306, and Li patent U.S. Pat. No. 6,610,079.

The disclosed system achieves an improved grip in delicate bone regions which is bioinert and less traumatic to the overlying soft tissues than prior art methods.

### Method of Use

A summary of the steps which a surgeon or health care provider performs to use expandable fixation devices to capture fracture fragments is as follows:

Using a closed, minimally invasive procedure or an open procedure, the target body region is accessed through a guide sheath. An incision is created in the skin (usually one incision, but a second small incision may also be required if a suction egress is used). Next, the bone or other tissue fragments are reduced to anatomic position, and, using a sheath for tissue protection, the bones (or pierce the soft tissues) to be fastened together are drilled to form a cavity or passage in the bone and/or other tissues. Next the area is measured to determine the appropriate inflatable fastener length and select the correct length biocompatible, bio-degradable inflatable balloon-like device. The device is inserted into the cavity or passage a pre-determined distance beyond the outer surface of the fragment(s) to be captured (visualize the position of the radio-opaque tip using fluoroscopy). Next the tip using a flowable biocompatible filling material, such as a synthetic bone substitute, is inflated. The cement expands slightly on hardening and compresses and captures the far fragment.

In one embodiment the hardened cement within the balloon fastens the fragments at both ends to each other and to any interposed fixation device (such as a bone screw or plate). In another embodiment, the inflation of the inflatable device causes a compacting of the cancellous bone and bone marrow against the inner surface of the cortical wall of the bone in addition to the outer edges of the bone or a periosteal plate. In yet another embodiment, sutures are used to achieve final tensioning, and then knotting or other securing means is applied after this tensioning. Following this latter step, the insertion instruments are removed from the body. Following the procedure, the incision is closed (sutured or stapled) and the wound is covered with a bandage.

The device can be pre-assembled for easy insertion and deployment, an example of which would be a catheter having a distal end sized and configured for insertion through a cannula into the tissues, The catheter contains at its distal end an collapsed, inflatable body configured for passage within the cannula into a drilled hole in bone or into a passage through soft tissue. When inflated the inflatable body is sized and configured to provide a fastening to one or more bone fragments, or to a plate or intramedullary nail. The shaped regions of the inflatable body allow capture of at least the outer surface of one bone or tissue fragment when inflated. The inflation size is predetermined by the balloon wall thickness, material properties and either the volume of filler injected or the pressure of filler injection.

The method includes a series of steps which a surgeon or health care provider can perform to form a cavity in pathological bone (including but not limited to osteoporotic bone, osteoporotic fractured metaphyseal and epiphyseal bone, osteoporotic vertebral bodies, fractured osteoporotic vertebral bodies, fractures of vertebral bodies due to tumors especially round cell tumors, avascular necrosis of the epiphyses of long bones, especially avascular necrosis of the proximal femur, distal femur and proximal humerus and defects arising from endocrine conditions).

To use the disclosed balloon with filler to align and hold tissue fragments together an incision is made in the skin (usually one incision, but a second small incision may also be required if a suction egress is used) followed by the placement of a guide pin that is passed through the soft tissue down to and into the bone.

The bone to be treated is drilled to form a cavity or passage in the bone, following which an inflatable balloon-like device is inserted into the cavity or passage and inflated.

A flowable biocompatible filling material, such as methylmethacrylate cement or a synthetic bone substitute, is then directed into the cavity or passage and allowed to set to a hardened condition to provide structural support for the bone. Following this latter step, the insertion instruments are removed from the body and the incision in the skin is covered with a bandage.

To reduce tissue trauma, it is advantageous to limit the number of surgical openings. Many unstable or comminuted fractures, particularly in the peri-articular regions near the ends of bones, require purchase on the outer surface of the bone to achieve optimal healing stability under desirable post operative load regimes.

The following is an example procedure for using a plug to keep the flexible membrane outside of bone while tensioning sutures and tissue (bone) fragments together. A pathway is created either by drilling through bone and/or opening tissue with cannula, needle or other means. The tube containing the expandable device is inserted into the opening and deployed, pushing the expandable end out of tube and beyond periosteal surface, The opening at the distal side is then blocked to prevent the membrane from retracting using a plug of metal, polymer, degradable polymer, ceramic polymer composite or injectable cement, typically containing tricalcium phosphate and/or hydroxyapatite ceramics). Tension is then applied to the connecting cord and, once the appropriate compression is achieved, the connecting cords are locked on the proximal side. The mechanism of locking is appropriate to the material to which the cords are locked. This can be a plate, screw, bone, suture button, knotless suture button, or other material that will be known to those skilled in the art. Locking mechanisms can include, but are not limited to, mechanical fasteners, polymeric or metallic; crimp, skewer, clamp melt, capture in snap-lock mechanism; or tying one or more knots in the tensioning bar or bars as required to achieve stability.

The membrane and cord combination is preferably pre-packaged in a tube to facilitate easy passage, and the tube may be part of a surgical tool, may be simple packaging material, or may be part of another implant such as a screw or peg which is seated in the tissue, and which may be affixed to or through a plate.

### Needs-Benefits

Increased control of hard-to-grip fragments (tension force sufficient to allow early passive ROM, or active ROM without resistance.)

Material that is atraumatic to moving ligaments, tendons, cartilage, and bone.

Relatively flat surface to prevent painful rubbing or impingement, and avoid motion blockage.

Sized to be used within cannulated screws, or pegs (tubes) which can be locked to plates or washers to take advantage of higher-strength higher-stiffness materials used in these devices and rigidity of locked connections.

When used in conjunction with traditional locked screws and plates, the device can provide buttressing (the ability to transfer loads to plate and withstand shear and bending at the plate interface, as opposed to merely providing compression of fragments via tension in the tensioning means.

Membrane and/or Balloon Material Examples:

The material must be non-cytotoxic, and the foreign body reaction minor. In this application it is preferable to have a slowly degrading material that does not swell extensively during degradation or result in extensive fibrous scar formation or a late inflammatory response as has been observed with the use of slowly degrading highly crystalline poly (L-lactide). It is also preferable for it to be somewhat flexible and strong. It must be easy to deploy, and yet strong enough to hold tissue fragments in place and distribute stresses over the membrane surface after deployment.

One such particularly useful membrane material is a slowly hydrolyzing (biodegradable) polycaprolactone-based polyurethane urea formulation as is manufactured by ArtImplant under the mark Artelon®, and other polymers fabrics or fibers such as polytrimethylene carbonate and poly (trimethylene-carbonate-co-e-caprolactone) copolymers which undergo slow degradation in vivo to non-toxic products. Artelon® a degradable polyurethaneurea, is a patented biomaterial that acts as a temporary support to the healing tissue. Excellent biocompatibility has been reported for the product.

Polycaprolactone (PCL) is degraded by hydrolysis of its ester linkages in physiological conditions (such as in the human body) and has therefore received a great deal of attention for use as an implantable biomaterial, in particular it is especially interesting for the preparation of long term implantable devices, owing to its degradation which is even slower than that of polylactide.

Another class of slowly hydrolyzing biocompatible membrane materials with good mechanical properties are manufactured from polyhydroxylkanoates, such as Metabolix, U.S. Pat. No. 6,878,758, and others.

Yet another material with suitable properties can be created from cross-linked collagen as described by Korb et al in U.S. Pat. No. 6,565,960

### Filler Material Examples:

The bioactive ceramic composite material biosorbability and reactivity is adjusted to meet the demands of bone plates and bone screws where the regional strength of the fastener is maintained either via reduced resorption or increased ossification for a period of about 1 to 2 years.

It is desirable to use a bone replacement material, which initially takes over the lost supporting function of the bone with high resistance to pressure, but then successively decreases in resistance to pressure, as a result of which the endogenous bone transformation processes (remodeling) are stimulated and hence more rapid osteoneogenesis and hence also active resorption of the bone replacement material is introduced. This may also be achieved by incorporating a slightly soluble substance, for example into a hardening cement paste. Because bone grows well into macroporous structures, it is advantageous to admix granular or pellet-like, solubilizing substances consisting of, for example sugars, salts (for example NaCl) or gypsum (CaSO.sub.4) into the cement paste. They are then leached out very rapidly in the body from the hardened cement structure and a porous sponge-like structure remains. The material must be workable for long period of time at room temperature, must be able to form may be formed at low temperatures, is readily formable and/or injectable, and yet can harden to high strength upon further reaction. To assure good fixation, the material cannot shrink as it consolidates. A material with low-grade expandable properties is also desirable.

Today, most surgeons use a calcium phosphate cement, in which initially a mechanically supporting mode of action is brought to bear, but the final resorption lags behind independently of the local transformation mechanisms of the bone, that is that the material is completely degraded. In addition, it is known in orthopedics that vital bone only remains where it is required from the biomechanical point of view. This is known as the so-called Wolff's Law. Consequently, if a calcium phosphate cement introduced into a bone defect has a higher compressive strength than the bone surrounding it and this high compressive strength remains unchanged, this leads to degradation of bone tissue lying around the implant (here calcium phosphate cement).

In order to fulfill this requirement, even if only partly, some manufacturers have admixed substances into their calcium deficient hydroxylapatites (CDHA) cements which are similar to nanoapatite, which are passively resorbed by the bodily fluids due to the concentration gradients, such as for example monetite (CaHPO.sub.4) or calcite (CaCO.sub.3) as known from European 0 543 765.

However, this only partly solves the problem. A cement is also required which can be resorbed completely passively and in which the resorption front and the deposition front are in direct contact.

One material with all of these features is found in Kyphon's Cement magnesium ammonium phosphate cement. (U.S. Pat. No. 6,908,506)

Another material with appropriate properties is a bioactive ceramic material that is biocompatible, bioresorbable and workable for long period of time at room temperature (Etex CaPO₄ Cement, Lee et al U.S. Pat. No. 6,953,594 This bioactive ceramic material contains poorly crystalline apatitic calcium phosphate solids with Ca/P ratios comparable to naturally occurring bone minerals and having stiffness and fracture toughness similar to natural bone.

### Cement Fillers:

The most important mineral constituents in human bone and tooth enamel are calcium and phosphate. However, considerable quantities of sodium, magnesium and carbonate are also present.

Calcium phosphates are not only biocompatible but are recognized by the living cell as belonging-to-the-body. Therefore, there are many biomaterials and medical products that consist partly of calcium phosphate. Calcium phosphate ceramics have been on the market since about 1970, partly in the form of prefabricated blocks or as granules. Implantations of these materials in bone structures are predominantly successful.

Calcium phosphate ceramics are most successful when they consist of hydroxylapatite (HA) or of beta-tertiary calcium phosphate (.beta-TCP, a whitlockite-like structure) or when the calcium phosphate ceramics consist of both, HA and beta.-TCP in variable ratios. HA is virtually non-resorbable from bone implantations, whereas beta.-TCP is slowly resorbed and replaced by new bone, It is therefore possible to influence the degree of resorption of calcium phosphate ceramic by changing the beta.-TCP/HA ratio.

It is likewise possible to admix other resorbable materials, such as: monetite CaHPO.sub.4, brushite CaHPO.sub.4-2H.sub.2O, calcite CaCasub.3 and dolomite CaMg (CO.sub.3).sub.2.

Since 1985 attempts have been made to develop calcium phosphate cements in order to avoid the disadvantages of prefabricated or granular-like calcium phosphate ceramics (W. E. Brown and L. C. Chow, “A new calcium phosphate, water-setting cement”, Cem. Res. Prog. 1986 352-379 (1987)).

This includes a brushite cement not yet commercially available having a Ca/P molar ratio of the precipitated phase of 1.00. This phase is not nanocrystalline but microcrystalline.

All the other calcium phosphate cements developed hitherto have a nanocrystalline precipitation structure and a Ca/P molar ratio of >=1.5, which may be further increased by addition of carbonate. These materials are known under U.S. Pat. No. 5,605,713; European application 0 835 668: World 96/14265, and some of these materials are on the market, However, there are contradictory reports regarding the resorbability of these materials after implantations in bone and soft tissue. In each case, calcium phosphate cements based on hydroxylapatite (HA) which are not resorbable (HA ceramics see above) and calcium phosphate cements based on deficient calcium hydroxylapatites (CDHA, calcium deficient hydroxylapatites) which are good osteotransductively, are differentiated. This means for the last-mentioned case, that they may be resorbed by osteoclasts and may be replaced by new bone tissue from osteoblasts. Resorption of these cements depends crucially on the local bone transformation mechanisms.

The deployment tubes used herein can be manufactured from metal, polymeric, degradable polymer, ceramic composite or ceramic-polymer composite or other materials that meet the criteria set forth herein.

All documents, patents, journal articles, and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

### BROAD SCOPE OF THE INVENTION

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive, For example, in the present disclosure, the term “preferably” is non-exclusive and means “preferably, but not limited to.”

In this disclosure and during the prosecution of this application, means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” may be used as a reference to one or more aspect within the present disclosure. The language present invention or invention should not be improperly interpreted as an identification of criticality, should not be improperly interpreted as applying across all aspects or embodiments (i.e., it should be understood that the present invention has a number of aspects and embodiments), and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure, the following abbreviated terminology may be employed: “e.g.” which means “for example”.

1- A method of securing biological materials comprising the steps of:
creating a passage through said biological materials, said passage having a distal end and a proximal end,
inserting a flexible connector through said passage, said flexible connector having a length greater than the length of said passage,
securing said connector to a first securing member, said first securing member being exterior of said passage distal end,
locking said connector external of said proximal end,
inserting said securing member, said securing member being a folded, flexible biocompatible membrane, and said flexible connector, into a deployment tube,
inserting said deployment tube into said passage,
deploying said membrane and said connector from said deployment tube to a position external said distal end of said passage,
removing said deployment tube,
locking said connector external of said proximal end.

2. (canceled)

3- The method of claim 1, further comprising said biological materials being at least one member of the group comprising bone fragments, tissue fragments, and combinations of bone and tissue fragments and comprising the step of applying a compressive force to fix bone fragments to bone, tissue to tissue, or bone and tissue fragments to bone.

4- The method of claim 1 further wherein said securing member is at least one member of the group comprising plate, flexible membrane, tensioning bar, and/or bone.

5- The method of claim 1 wherein said flexible membrane is bioincorporable.

6- The method of claim 1, wherein said connector material is at least one member of the biocompatible material group comprising resorbable and non-resorbable materials, woven cords, fibers, strands, K-wires, polymeric materials, composites, and metallic cables, wires, or rods.

7- The method of claim 1, wherein said connector is a tensioning member, said tensioning member applying compressive force to said biological material.

8- The method of claim 7, further comprising the step of tying said connector to a plate and securing said plate to bone, or tissue fragments, or combinations thereof with said tensioning member.

9- The method of claim 7, further comprising a block tube, said block tube being dimensioned to receive said deployment tube, and comprising the steps inserting said block tube into said passage, deploying said flexible membrane external of said passage and removing said deployment tube, leaving said flexible membrane external of said passage.

10- The method of claim 7, wherein said connector member is a suture looped through said first membrane and further comprising the step of tying off said connector at the proximate end of said passage.

11- The method of claim 10, wherein said tying of said fastener comprising the step of fixing said fastener proximal ends with a locking means.

12-30. (canceled)
